SEQUENCE LISTING (6 sequences)

SEQ ID NO: 1 - DNA, 19 bp, synthetic construct
  misc_feature 1..19: note = THBS1
  source 1..19: mol_type = other DNA
agactccgcatcgcaaagg

SEQ ID NO: 2 - DNA, 21 bp, synthetic construct
  misc_feature 1..21: note = THBS1
  source 1..21: mol_type = other DNA
tcaccacgttgttgtcaaggg

SEQ ID NO: 3 - DNA, 20 bp, synthetic construct
  misc_feature 1..20: note = THBS3
  source 1..20: mol_type = other DNA
ctcggcagatggtagctgtg

SEQ ID NO: 4 - DNA, 23 bp, synthetic construct
  misc_feature 1..23: note = THBS3
  source 1..23: mol_type = other DNA
tggataagaggtagatgtcccca

SEQ ID NO: 5 - DNA, 21 bp, synthetic construct
  misc_feature 1..21: note = GAPDH
  source 1..21: mol_type = other DNA
ggagcgagatccctccaaaat

SEQ ID NO: 6 - DNA, 23 bp, synthetic construct
  misc_feature 1..23: note = GAPDH
  source 1..23: mol_type = other DNA
ggctgttgtcatacttctcatgg

(19) United States
(12) Patent Application Publication
(10) Pub. No.: US 2023/0273224 A1
(43) Pub. Date: Aug. 31, 2023

(54) APPLICATION OF THBS1 PROTEIN AND THBS3 PROTEIN AS BIOMARKERS FOR DETECTING ACUTE MYOCARDIAL INFARCTION

(71) Applicant: Jilin University, Changchun (CN)

(72) Inventors: Heyu Meng, Changchun (CN); Jianjun Ruan, Changchun (CN); Fanbo Meng, Changchun (CN); Yanqiu Chen, Changchun (CN); Zhaohan Yan, Changchun (CN)

(21) Appl. No.: 18/050,640

(22) Filed: Oct. 28, 2022

(30) Foreign Application Priority Data
Dec. 31, 2021 (CN) 2021116638972

(51) Int. Cl. G01N 33/68

(52) U.S. Cl. CPC G01N 33/6893; G01N 2800/324

(57) ABSTRACT

An application method of at least one of a THBS1 protein and a THBS3 protein as biomarkers for detecting acute myocardial infarction is provided, which belongs to the field of biomedical technology. The THBS1 protein and THBS3 protein in peripheral plasma are low expressed in patients with acute myocardial infarction through proteomic results, the proteomic results are verified by expanding the number of patients’ samples, and the two proteins can be used as molecular markers for the diagnosis of acute myocardial infarction. It has the advantages that: (1) peripheral blood samples are easy to obtain, saving time and labor; (2) the experiment is simple and easy to operate; (3) a new intervention target for diagnosis and treatment of acute myocardial infarction is found; (4) a new direction is provided for treating acute myocardial infarction and improving prognosis in the future; and (5) a new idea is provided for precision medicine.

### TECHNICAL FIELD

The disclosure relates to the field of biomedical technologies, and more particularly to a use/application of a thrombospondin-1 (THBS1) protein and a thrombospondin-3 (THBS3) protein as biomarkers for detecting acute myocardial infarction.

### STATEMENT REGARDING SEQUENCE LISTING

The sequence listing associated with this application is provided in text format in lieu of a paper copy and is hereby incorporated by reference into the specification. The name of the XML file containing the sequence listing is 22082TBYX-USP1-MF-2022-0085-SL.xml. The XML file is 3,286,769 bytes; was created on Oct. 24, 2022; contains no new matter; and is being submitted electronically via EFS-Web.

### BACKGROUND

Cardiovascular disease is the main cause of morbidity and mortality in humans. Acute myocardial infarction (AMI) is one of the most serious cardiovascular diseases in the world. In addition, AMI is also considered to be one of the main causes of disability and mortality of cardiovascular diseases worldwide, and a major threat to human health. In recent years, the incidence of cardiovascular diseases has continued to rise, and at present, the cardiovascular disease deaths account for the first cause of death among urban and rural residents. Therefore, it is urgent to improve the prevention and treatment of AMI.

AMI is a result of interaction of many factors, including genetic and environmental factors. In addition, the incidence of AMI shows obvious familial aggregation, and genetic factors play an important role in its occurrence and development. Therefore, early acquisition of accurate genetic biomarkers of acute myocardial infarction can enable patients to make early diagnosis in time, so as to make correct treatment decisions. Therefore, it is important to find new molecular markers for early diagnosis, early warning, early intervention and improvement of prognosis of AMI.

### SUMMARY

A purpose of the disclosure is to provide a use/application of a thrombospondin-1 (THBS1) protein and a thrombospondin-3 (THBS3) protein as biomarkers for detecting acute myocardial infarction, so as to solve problems existing in the related art. By detecting expressions of the THBS1 protein and the THBS3 protein in patients’ peripheral plasma, it can provide a reliable basis for early diagnosis and timely treatment of AMI patients, and provide an intervention target for future treatment of diseases.

To achieve the above purpose, the disclosure provides the following solutions.

Specifically, the disclosure provides an application method of at least one of aTHBS1 protein and a THBS3 protein, including: using the at least one of the THBS1 protein and the THBS3 protein to prepare a diagnostic product of acute myocardial infarction.

The THBS1 protein and the THBS3 protein belong to the thrombospondin (THBS) family. Both the THBS1 protein and the THBS3 protein are glycoproteins, which can participate in many processes of vascular wall, including smooth muscle cell proliferation, and endothelial cell proliferation and migration. The proteomic results of the disclosure show that the THBS1 protein and THBS3 protein in peripheral plasma are low expressed in patients with acute myocardial infarction, and the proteomic results of the THBS1 protein and THBS3 protein are verified by expanding the number of patients’ samples, so that the above two proteins can be used as molecular markers for diagnosing acute myocardial infarction.

In an embodiment, the at least one of the THBS1 protein and the THBS3 protein is configured as biomarkers.

In an embodiment, the diagnostic product of the acute myocardial infarction is used for at least one of determination of acute myocardial infarction, selection of therapy, and prognostic evaluation.

In an embodiment, the diagnostic product of the acute myocardial infarction includes a detection reagent of the acute myocardial infarction, and the detection reagent of the acute myocardial infarction is a reagent specifically detecting the at least one of the THBS1 protein and the THBS3 protein.

In an embodiment, the at least one of the THBS1 protein and the THBS3 protein is derived from peripheral blood plasma.

In an embodiment, the at least one of the THBS1 protein and the THBS3 protein is low expressed in patients with the acute myocardial infarction.

The disclosure also provides an acute myocardial infarction detection kit, including a detection reagent of the acute myocardial infarction, and the detection reagent of the acute myocardial infarction is a reagent specifically detecting at least one of a THBS1 protein and a THBS3 protein.

In an embodiment, the detection kit of acute myocardial infarction is an enzyme-linked immunosorbent assay (ELISA) detection kit.

In an embodiment, the reagent specifically detecting the at least one of the THBS1 protein and the THBS3 protein includes: at least one of a human THBS1 protein capture antibody and a human THBS3 protein capture antibody.

The disclosure discloses technical effects as follows.

The disclosure has the advantages that: (1) peripheral blood samples are easy to obtain, saving time and labor; (2) the experiment is simple and easy to operate; (3) a new intervention target for diagnosis and treatment of acute myocardial infarction is found; (4) a new direction is provided for treating acute myocardial infarction and improving prognosis in the future; and (5) a new idea is provided for precision medicine.

### BRIEF DESCRIPTION OF DRAWINGS

In order to explain embodiments of the disclosure or technical solutions in the related art more clearly, the following will briefly introduce the drawings needed in the embodiments. Apparently, the drawings in the following description are only some embodiments of the disclosure, and those skilled in the art can also obtain other drawings based on these drawings without paying creative labor.

FIG. 1 illustrates a standard curve of thrombospondin-1 (THBS1) protein.

FIG. 2 illustrates a standard curve of thrombospondin-3 (THBS3) protein.

FIG. 3 illustrates expression of the THBS1 protein.

FIG. 4 illustrates expression of the THBS3 protein.

FIG. 5 illustrates a receiver operator characteristic (ROC) curve of the THBS1 protein.

FIG. 6 illustrates a ROC curve of the THBS3 protein.

FIG. 7 illustrates reverse transcription-polymerase chain reaction (RT-PCR) amplification curves of THBS 1 and THBS3 genes in peripheral blood leukocytes.

FIG. 8 illustrates RT-PCR dissolution curves of the THBS1 and THBS3 genes in peripheral blood leukocytes.

FIG. 9 illustrates THBS1 proteomics results.

FIG. 10 illustrates THBS3 proteomics results.

FIG. 11 illustrates a protein heatmap, where T2 represents a stable coronary heart disease group, T3 represents an acute myocardial infarction group, and C1 represents a healthy group.

### DETAILED DESCRIPTION OF EMBODIMENTS

Various illustrative embodiments of the disclosure are described in detail, and the detailed description should not be considered as a limitation of the disclosure, but should be understood as a more detailed description of some aspects, features, and embodiments of the disclosure.

It should be understood that terms described in the disclosure are only used to describe particular embodiments and are not intended to be limiting of the disclosure. In addition, with respect to numerical ranges in the disclosure, it should be understood that each intermediate value between upper and lower limits of the range is also specifically disclosed. Each smaller range between intermediate values within any stated values or stated range and intermediate values within any other stated values or the range is also included in the disclosure. The upper and lower limits of these smaller ranges can be independently included or excluded from the range.

Unless otherwise stated, all technical and scientific terms used herein have the same meanings as those commonly understood by those skilled in the art of the disclosure. Although the disclosure only describes preferred methods and materials, any methods and materials similar or equivalent to those described herein may also be used in the implementation or testing of the disclosure. All documents mentioned in the specification are incorporated by reference to disclose and describe methods and/or materials related to the documents. In case of conflict with any incorporated documents, the contents of the specification shall prevail.

It will be apparent to those skilled in the art that various modifications and variations can be made in the specific embodiments described herein without departing from the scope or spirit of the disclosure. Other embodiments obtained from the specification of the disclosure will be apparent to those skilled in the art. The specification and embodiments of the disclosure are only illustrative.

As used herein, the terms “comprising”, “including”, “having”, “containing” and the like used herein are all open terms, meaning including but not limited to.

### Embodiment 1

### 1. Experimental Objects

In this study, 112 patients with acute myocardial infarction (AMI) hospitalized in the Department of Cardiology, China-Japan Friendship Hospital of Jilin University from January 2017 to January 2019 were selected as an experimental group.

The diagnosis of AMI is based on the global definition of myocardial infarction issued by the European Society of Cardiology in 2017, that is, coronary angiography confirmed that the existence of definite vascular lesions with severe stenosis and occlusion of main coronary arteries (left coronary artery and right coronary artery, etc.) and main branches (circumflex branch and anterior descending branch, etc.).

The exclusion criteria of AMI are as follows: (1) myocardial infarction secondary to ischemic imbalance; (2) myocardial infarction in which serum biochemical markers (troponin and myoglobin) cannot be obtained; (3) myocardial infarction related to percutaneous coronary intervention or in-stent thrombosis; and (4) myocardial infarction related to coronary artery bypass grafting.

111 patients with stable coronary artery disease (SCAD) were selected as a control group. The inclusion criteria of SCAD were in line with the Canadian Cardiovascular Society’s grade 4 criteria for defining SCAD as follows. In grade I, general physical activity (such as walking and climbing stairs) is limited, but angina pectoris occurs during intense, fast, or sustained exercise. In grade II, general physical activity is slightly limited, and angina pectoris occurs during brisk walking, after meals, under cold conditions, during mental stress, or within a few hours after waking up. Generally, physical activity is limited to walking more than 200 meters (m) or climbing a flight of stairs. In grade III, general physical activity is significantly limited, walking within 200 m or climbing a flight of stairs causes angina pectoris. In grade IV, angina pectoris may occur during mild activity or rest. In the SCAD group, patients with SCAD received routine color Doppler echocardiography to exclude cardiac thrombosis and heart failure.

### 2. Experimental Methods

The peripheral blood of the patients in the experimental group and the control group was collected after admission and put into anticoagulant tubes containing ethylenediaminetetraacetic acid (EDTA). The plasma was separated by centrifugation within 30 minutes after collection and stored in a refrigerator at -80° C.

Subsequent experiments were performed according to procedures of an enzyme-linked immunosorbent assay (ELISA) kit (purchased from Shanghai Enzyme-linked Biotechnology Co., Ltd.). The operation process was as follows:

Step (1), the required plates were taken out from the aluminum foil bag equilibrated at room temperature for 60 minutes, and the remaining plates were sealed with a self-sealing bag and put them back a refrigerator at 4° C.

Step (2), standard wells, blank wells and sample wells were set, and 50 microliters (µL) of standard samples with different concentrations were added respective standard wells.

Step (3), 50 µL of the sample to be tested was added into the sample well, and 50 µL of a sample diluent was added into the blank well.

Step (4), 100 µL of horseradish peroxidase (HRP) labeled antibody was added into each well of the blank well, the standard well and the sample well, and the reaction wells were sealed with SealPlate® film and incubated at 37° C. for 60 minutes.

Step (5), the liquid was discarded, the well plate was patted dry on the bibulous paper, and each well was filled with a washing solution (350 µL), stood for 1 minute, shaken off the washing solution, patted dry with bibulous paper, and repeated for 5 times.

Step (6), 50 µL of substrate A and 50 µL of substrate B were added to each well and incubated in dark at 37° C. for 15 minutes.

Step (7), 50 µL of stop solution was added into each well, measured an optical density (OD) value of each well at the wavelength of 450 nanometers (nm) within 15 minutes.

A standard curve was drawn according to OD values of the blank well and the standard well, and a protein concentration of the sample was obtained by using the standard curve . Each sample of the experimental group and the control group was repeated 3 times.

### 3. Statistical Analysis Methods

SPSS25.0 statistical software was used for data analysis. Independent-t-test and rank sum test were used for comparison between groups. The measurement data conforming to normal distribution are expressed as mean ± standard deviation, and the measurement data not conforming to normal distribution are expressed as median and quartile. The risk factors of AMI were analyzed by using binary logistic regression analysis.

### 4. Results

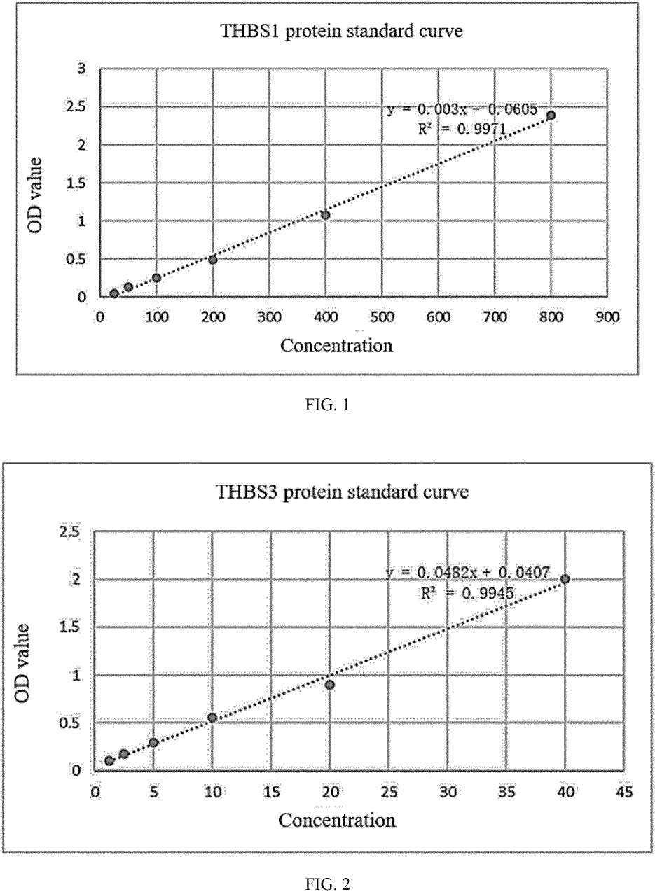

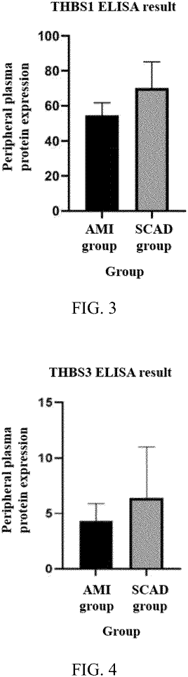

Standard curves of the THBS1 protein and the THBS3 protein are shown in FIG. 1 and FIG. 2. The results of ELISA are shown in FIG. 3 and FIG. 4.

The results of ELISA showed that the expression of THBS1 protein in patients with AMI was 0.79 times that of the SCAD group (P=0.000), and the expression of THBS3 protein was 0.82 times that of SCAD group (P=0.000). Both THBS1 and THBS3 showed low expression in AMI. The binary logistic regression analysis showed that the low expression of THBS1 and THBS3 at a protein level in human peripheral plasma was an independent risk factor for AMI.

### 5. Test Sensitivity and Specificity of THBS1 and THBS3 Protein Markers

### (1) Complementary Deoxyribonucleic Acid (cDNA) Synthesis of Peripheral Blood Lymphocytes

The Total ribonucleic acid (RNA) Blood Kit is used to extract total RNA from lymphocytes. In order to avoid RNA degradation or contamination, the extraction process shall be carried out in strict accordance with the instructions of the kit. The quality of RNA solution was detected by polyacrylamide gel electrophoresis. The 28 S and 18 S rRNA bands were visible, and the brightness of the 28 S rRNA band was about twice that of the 18 S rRNA band. The concentration and absorbance of the standard sample were determined by microplate reader. After meeting the requirements, reverse transcription was carried out.

Reverse transcription was performed on the total RNA that met the experimental requirements according to the instructions of the reverse transcription kit, and a consistent concentration of RNA was added to each sample. DNA samples obtained were stored at - 80° C. for subsequent fluorescent, quantitative, polymerase chain reaction assays.

### (2) Detection of Reverse Transcriptase Polymerase Chain Reaction (RT-PCR)

After the obtained DNA samples were diluted 20 times, PCR amplification was performed using the SYBR fluorescence quantitative kit. Glyceraldehyde-3-phosphate dehydrogenase (GAPDH) was used as the reference gene, and free fatty acid receptor 2 (FFAR2) was used as the target gene. The specificity of the amplification conditions was determined by using the software dissolution curve of the ABI-FAST7500 instrument. The sequences of the RT-PCR primers used are shown in the following table:

| | | |
|---|---|---|
| | Gene | Gene primer sequence (5′-3′) |
| THBS1 | F^{a} | AGACTCCGCATCGCAAAGG (SEQ ID NO: 1) |
| | R^{b} | TCACCACGTTGTTGTCAAGGG (SEQ ID NO: 2) |
| | F^{a} | CTCGGCAGATGGTAGCTGTG (SEQ ID NO: 3) |
| THBS3 | R^{b} | TGGATAAGAGGTAGATGTCCCCA (SEQ ID NO: 4) |
| | F^{a} | GGAGCGAGATCCCTCCAAAAT (SEQ ID NO: 5) |
| GAPDH | R^{b} | GGCTGTTGTCATACTTCTCATGG (SEQ ID NO: 6) |

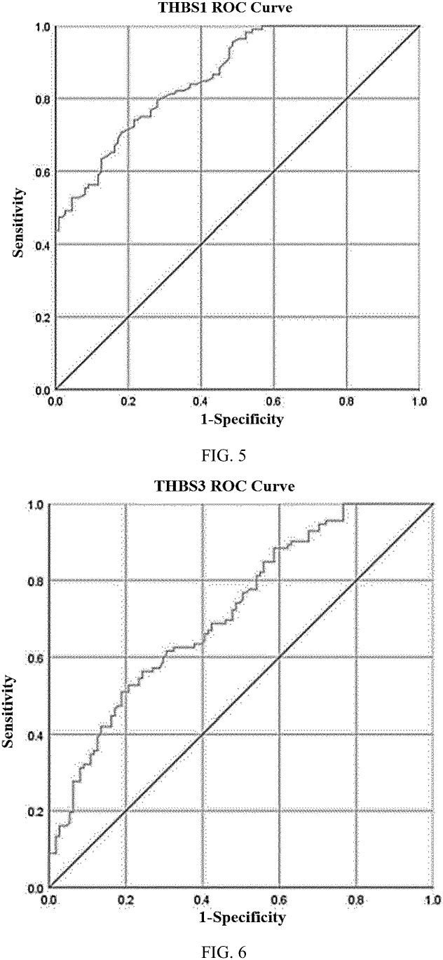

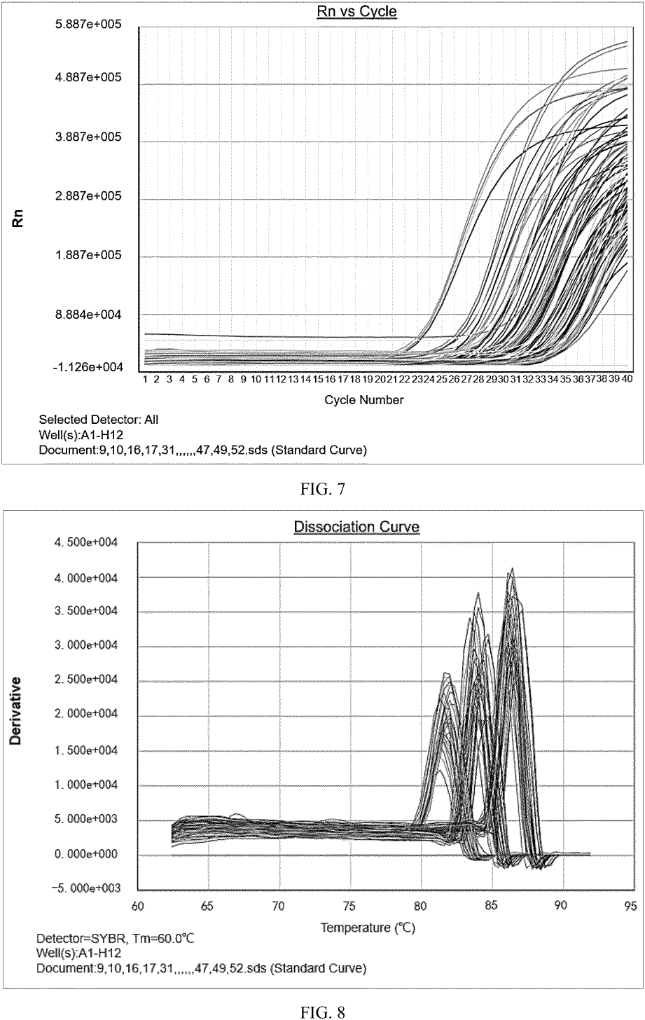

RT-PCR experiment results are shown in FIGS. 5-8, and the statistics of receiver operator characteristic (ROC) results are shown in the following table:

| | AUC | Standard error | Asymptotic significance | Asymptotic 95% confidence interval | Sensitivity | Specificity |
|---|---|---|---|---|---|---|
| THBS1 | 0.861 | 0.023 | 0.000 | 0.816-0.907 | 0.705 | 0.820 |
| THBS3 | 0.718 | 0.034 | 0.000 | 0.652-0.783 | 0.527 | 0.793 |

Gene analysis of THBS1 and THBS3 as follows. Specifically, identification of RT-PCR products: amplification curves of the reference gene, THBS1 gene, and THBS3 gene each are an obviously smooth S-shaped curve, and dissociation curves each is a single peak without multiple peaks, indicating that the amplified primers have strong specificity, the reaction conditions are appropriate, and there is no non-specific amplification. Analysis of THBS1 and THBS3 gene expression level: RT-PCR was repeated three times for each sample, and the standard deviation meets the requirements of RT-PCR. The AMI group and the control group were tested by independent sample t-test, which met the requirement of p < 0.05.

The results showed that the relative expression of THBS1 gene between the AMI group and the control group (i.e., 2-ΔCt value measured by PCR quantification) was 0.054 (0.030-0.074) and 0.132 (0.049-0.310), respectively. There was significant difference between the two groups (p<0.05). The relative expression of THBS1 gene in peripheral blood of AMI patients was significantly lower than that of the control group (0.41 times). The relative expression of THBS3 gene between the AMI group and the control group (i.e., 2-ΔCt value measured by PCR quantification) was 0.014 (0.009-0.039) and 0.030 (0.019-0.098), respectively. There was significant difference between the two groups (p<0.05). The relative expression of THBS3 gene in peripheral blood of AMI patients was significantly lower than that of the control group (0.47 times).

### Embodiment 2 Proteomics Experiment

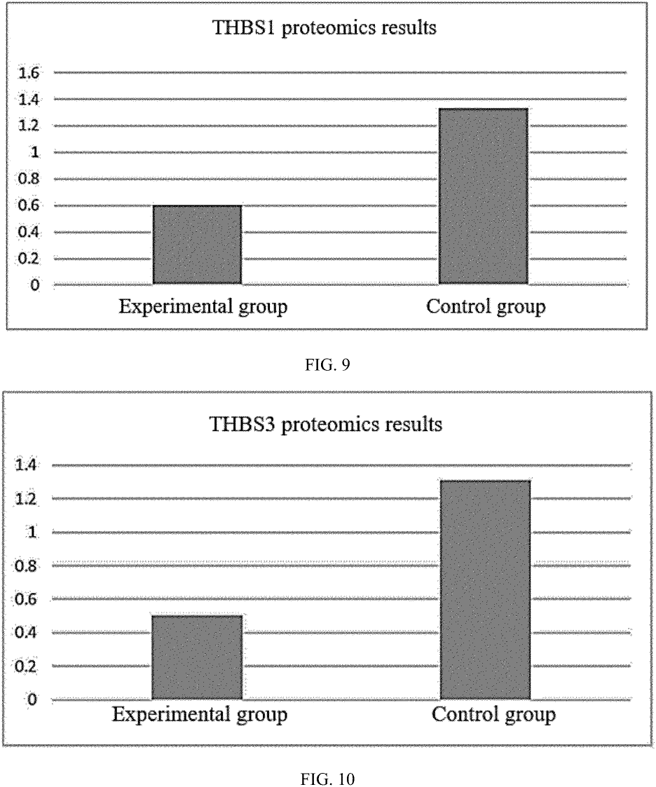

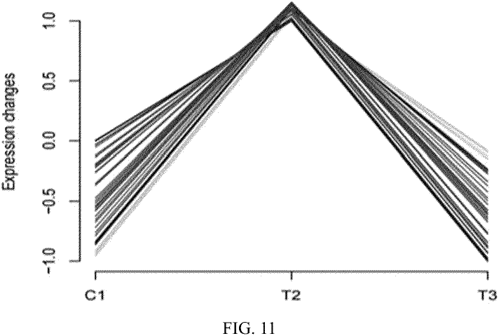

According to gender and age, patients were randomly divided into healthy group, stable coronary heart disease control group and acute myocardial infarction experimental group respectively. Tandem mass marker (TMT) quantitative proteomics technology was used to analyze the changes of protein spectrum of peripheral blood plasma samples, and the differentially expressed proteins were screened. Subsequently, by expanding the sample size, 19 patients in the stable coronary heart disease control group and 20 patients in the acute myocardial infarction experimental group were selected, and the parallel reaction monitoring (PRM) experiment was used to verify and analyze their biological functions. The results are shown in FIGS. 9-11.

It can be seen from FIGS. 9-11 that there is a significant difference in THBS1 and THBS3 proteins between the stable coronary heart disease control group and the acute myocardial infarction experimental group, and the differences are statistically significant.

The above-described embodiments only describe the preferred mode of the disclosure, do not limit the scope of the disclosure. Without departing from the design spirit of the disclosure, all modifications and changes made by those skilled in the art to the technical solution of the disclosure shall fall within the scope of protection defined in appended claims of the disclosure.

What is claimed is:

1. An application method of a reagent specifically detecting a thrombospondin-1 (THBS1) protein and a thrombospondin-3 (THBS3) protein, comprising:
using the reagent specifically detecting the THBS1 protein and the THBS3 protein to design or prepare a diagnostic product, wherein the diagnostic product is applied for distinguishing acute myocardial infarction and stable coronary heart disease (SCHD),
wherein the THBS1 protein and the THBS3 protein is derived from peripheral blood plasma.

2-5. (canceled)

6. The application method according to claim 1, wherein the THBS1 protein and the THBS3 protein is low expressed in patients with the acute myocardial infarction.

7-9. (canceled)
